(19) United States
(12) Patent Application Publication
(10) Pub. No.: US 2013/0041374 A1
(43) Pub. Date: Feb. 14, 2013

(54) RADIUS INTRAMEDULLAR LOCKING NAIL

(71) Applicant: Fuat Akpinar, Istanbul (TR)

(72) Inventor: Fuat Akpinar, Istanbul (TR)

(21) Appl. No.: 13/643,299

(22) PCT Filed: Aug. 3, 2010
(86) PCT No.: PCT/TR10/00161

§ 371 Date: Oct. 25, 2012

(30) Foreign Application Priority Data
Apr. 27, 2010 (TR) 2010/03304

(51) Int. Cl. A61B 17/72

(52) U.S. Cl. 606/64

(57) ABSTRACT

This invention is related to the radius intramedullar locking nail which provides fusion by fixing the bone in case of single or multiple fractures of radius bone, which fixes the distal end of the nail with a locking screw (5) and the other end of it with the bayonet end of the elastic nail (3) by applying it through the medulla of radius bone and by taking the broken bone part in the middle; and its feature is characterized by consisting of that radius intramedullar elastic nail (1), locking distal hole (2), the bayonet end of the elastic nail (3), anatomic curves of the elastic nail (4), locking, conical headed cortical bone screw (5), holed oval head of the elastic radius nail (6), inserter of the elastic radius nail (7), on-off button of the inserter (8) and the impacter of the inserter (9).

### TECHNOLOGICAL SCOPE

This invention is related to the radius intramedullar locking nail which provides fusion by fixing the bone in case of single or multiple fractures of radius bone, which fixes one end of the nail with a screw by applying it through the medulla of radius bone and by to taking the broken bone part in the middle.

### COMMON STATUS OF THE TECHNIQUE

Radius is one of the front arm bones.

At the present time, plate-screw fixation for radius bone fractures is widely used. Many complications can be faced with in patients who this method has been applied. So the ideal point hasn't been able to reach yet in the nail systems which has been developed and put into practice in the last years with the thought of that complications will be reduced to the minimum with appropriate intramedullar nail development.

As for the radius intramedullar locking nail, it's in the way of fixing the radius bone fractures with an intramedullar nail application and being knitted in time. The bone's compression and fusion are provided by fixing the nail below the bone. The radius nail provides compression and fusion of the broken bone by placing it in the radius bone.

An implant which will be able to treat all type of radius bone fractures very well hasn't been existed in the present technology.

This invention is related to the radius intramedullar locking nail that can overcome the disadvantages determined above, and it's feature; is to be a special easy applied design that provides locking on the below part of the intramedullar nail in the system.

### EXPLANATION OF THE FIGURES

This invention has been described in more details just by illustrating it by referring to the attached drawings after this, on these drawings;

FIG. 1 Perspective view of the radius nail

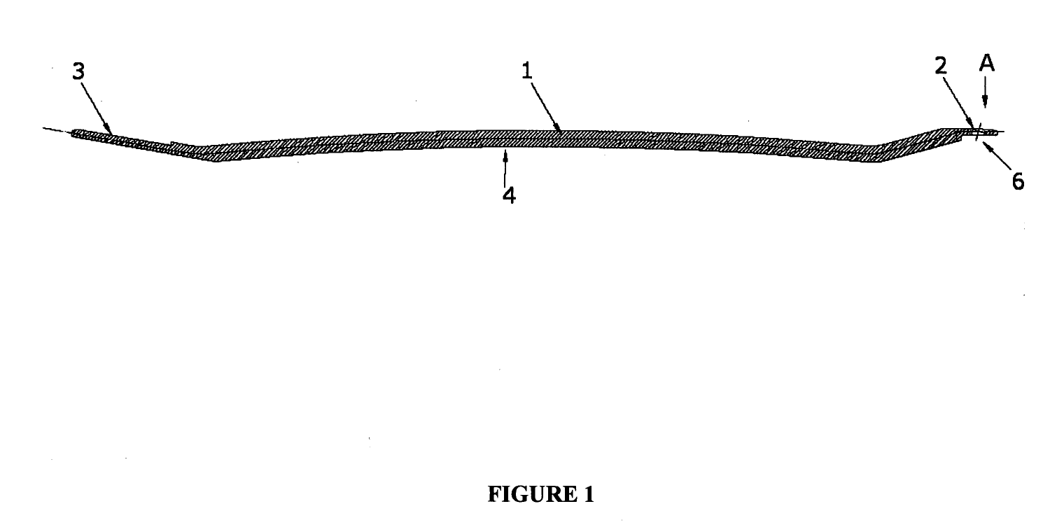

FIG. 2 Assembly view of the radius nail with the inserter apparatus

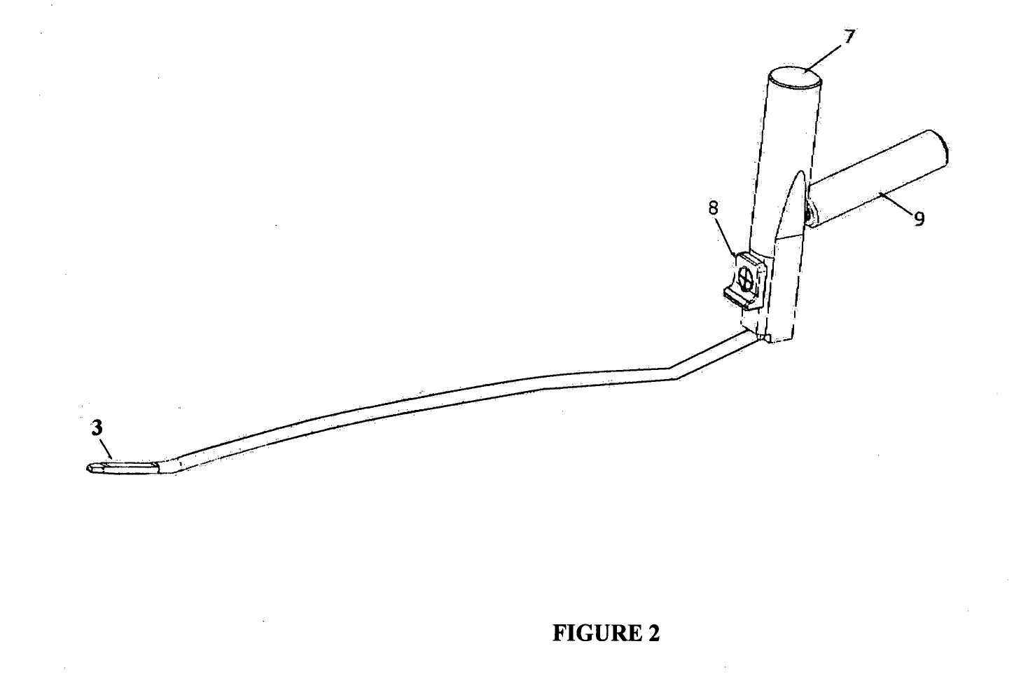

FIG. 3 The view of the radius nail from above

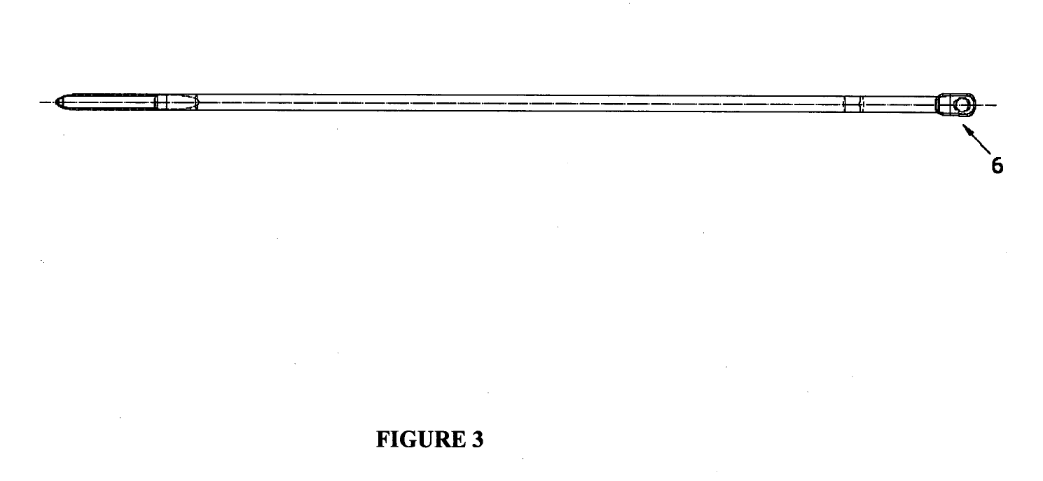

FIG. 4 Locking screw view providing the distal locking of the nail

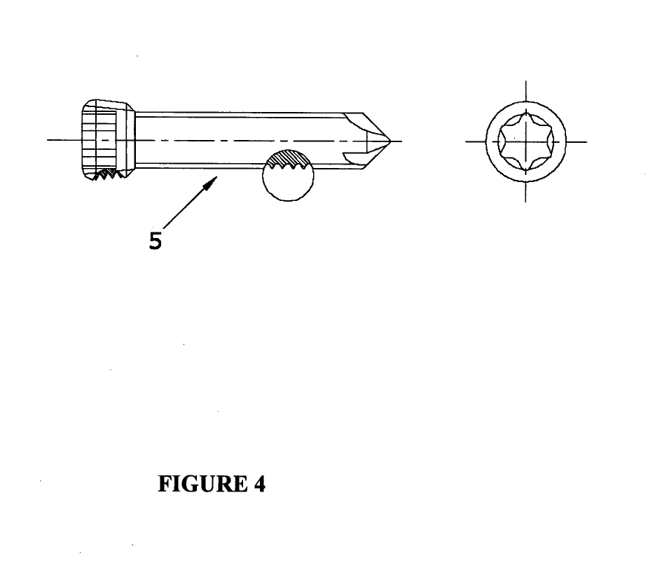

FIG. 5 View from A. The view of holed oval head of the elastic radius nail

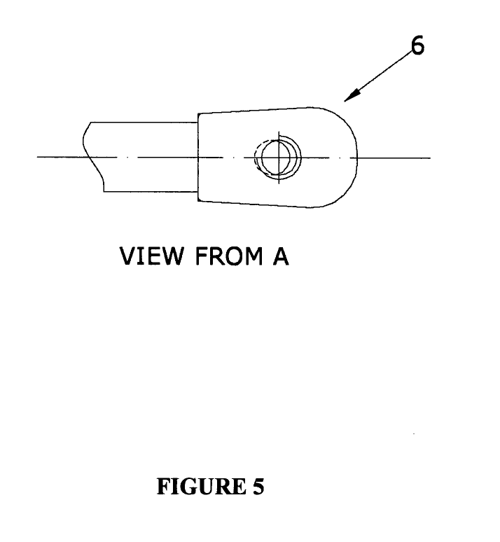

The figures which will help this invention to be understood have been numbered as determined on the attached drawings and these numbers have been shown together with their names in the following.

### DESCRIPTION OF THE REFERENCES

1. Radius intramedullar elastic nail
2. Locking distal hole
3. The bayonet end of the elastic nail
4. Anatomic curves of the elastic nail
5. Locking, conical headed cortical bone screw
6. Holed oval head of the elastic radius nail
7. Inserter of the elastic radius nail
8. On-off button of the inserter
9. The impacter of the inserter

### DESCRIPTION OF THE INVENTION

This invention is related to the radius intramedullar locking nail, and its feature is characterized by consisting of that radius intramedullar elastic nail (1), locking distal hole (2), the bayonet end of the elastic nail (3), anatomic curves of the elastic nail (4), locking, conical headed cortical bone screw (5), holed oval head of the elastic radius nail (6), inserter of the elastic radius nail (7), on-off button of the inserter (8) and the impacter of the inserter (9).

The aim of the invention:
To reduce the number of surgical instruments to be used during operation
To be able to insert and extract easily and so to shorten the operation time.
To provide the maximum resistance necessary against the rotational and bending forces
To be able to make locking easily on both ends (proximal and distal) of the bone by removing or reducing to the minimum the need of scopy and guide used in the previous technologies.
To eliminate the screw migration on the ends.
To prevent screw and nail breakages
To provide the most possible stable fixation with the nail (1) that has been produced from the material which its elasticity is the most similar to the bone
To prevent the stress fractures that could be existed as the fixation material is in the bone
To reduce re-breakage risk to the minimum after the fixation material is extracted.
To provide intramedullar fixation eliminating the need for external fixation and allowing early movement, being able to be placed through the whole bone.

The features of the radius nail (1) are; being solid, round, unreamed, conic ended, having titanium flexibility and biocompatibility.

3-4 cm part of the proximal is 8 to 14 degrees anterior angled and oblong. Diameter choices as for the distal part are 3 to 4 mm; length choices for the nail are 17 to 25 cm and, 3-4 cm part of the distal is 15 degrees angled to the front, there exists one hole 16 to 18 degrees angled on the distal end for the locking screws 2.3 mm to 2.7 mm.

The body part is parabolic (1 cm R). The same nail (1) is used for right and left bones. There is no need for usage of scopy or guide for distal and proximal locking.

It has a new, ergonomic and simple inserting and locking system. It won't need a special set for extraction.

Owing to the proximal design of the nail (1), rotational stabilization is provided on itself during proximal locking. When required, stability could be increased with a poller screw or a set screw.

As for the distal locking, interlocking is made with one 2.3 to 2.7 mm screw owing to the 16 to 18 degrees prox-voler angled oblique hole existing on the distal part of the nail. Locking screws have diameters 2.3 to 2.7, and they have 7 different length choices as 12, 14, 16, 18, 20, 22, 24 mm and there is no need for usage of scopy or guide for their insertion.

The invention is used for the followings on the region from 2 cm distal of radius proximal joint surface to the proximal of distal radio-ulnar joint;
For all radius fractures
In cases of malunion or non-union
For Galeazzi dislocations with fractures
For Colles fractures (for distal methaphysis fractures having single fracture line not being related to radio-ulnar and radio-carpal joint)
For shortening osteotomies
For tumor resections

As applying this invention, two-sided graphy from 1 meter distance of the healthy front arm is taken before operation. Probable nail diameter to be used is determined by measuring the diameter of the narrowest medullar region; and the nail length is determined by measuring the distance between the joint surfaces and by subtracting 3 cm from it. The nails (1) having diameters and lengths one size smaller and bigger than the measured diameter and length of the nail are prepared. 2 cm incision is made to the proximal on the wrist dorsal radial of the patient prepared for the operation. It's directed to medulla on methaphysis by opening an entrance hole with a drill from radius styloid or tunel 2. The nail (1) prepared with the nail holder beforehand is moved ahead from the entrance hole towards the proximal with partial rotations. If reduction could be provided by hand; when the nail (1) is moved ahead, proximal fragment is passed to. If reduction couldn't be provided; the nail (1) is sent to the most proximal as possible by following the reduction with scopy control, and with mini incision if the reduction doesn't happen again.

The nail (1) is locked through its distal hole (2) with a screw having diameter 2.3 to 2.7 mm. Proximal stability is provided easily owing to the nail's special anatomic design. If required, stability can be increased with a poller screw or a setscrew. It's allowed to make movements of hand, wrist, front arm and elbow in the earliest time after operation.

1. This invention is the radius intramedullar locking nail which provides fusion by fixing the bone in case of single or multiple fractures of radius bone, which fixes the distal end of the nail with a locking screw (5) and the other end of it with the bayonet end of the elastic nail (3) by applying it through the medulla of radius bone and by taking the broken bone part in the middle; and its feature is characterized by consisting of that radius intramedullar elastic nail (1), locking distal hole (2), the bayonet end of the elastic nail (3), anatomic curves of the elastic nail (4), locking, conical headed cortical bone screw (5), holed oval head of the elastic radius nail (6), inserter of the elastic radius nail (7), on-off button of the inserter (8) and the impacter of the inserter (9).

2. This invention is the radius intramedullar locking nail (1); and its feature is characterized by providing proximal locking by the aid of the bayonet end (3) owing to the nail (1) design, and by providing rotational stability with the holed oval head (6) of the nail being locked through the locking distal hole (2) with locking, conical headed cortical bone screw (5).
